(19) United States
(12) Patent Application Publication
(10) Pub. No.: US 2005/0119583 A1
(43) Pub. Date: Jun. 2, 2005

(54) Heart monitor

(76) Inventors: Jonathan Andrew Fuller, Iverness (GB); Daniel Strother, Inverness (GB); Nigel Alastair Buist Simpson, Leeds (GB)

Correspondence Address: SUGHRUE MION, PLLC, 2100 PENNSYLVANIA AVENUE, N.W., SUITE 800, WASHINGTON, DC, 20037, US

(21) Appl. No.: 10/498,940

(22) PCT Filed: Dec. 16, 2002
(86) PCT No.: PCT/GB02/05692

(30) Foreign Application Priority Data
Dec. 22, 2001 (GB) 0130906.1

(51) Int. Cl. A61B 5/04

(52) U.S. Cl. 600/511; 600/372; 600/391

(57) ABSTRACT

A heart rate monitor apparatus and method are described. The apparatus consists of multiple recording electrodes each incorporated into a contact surface for application to an external body surface of a human or animal subject and a data acquisition and processing system to process the data by way at least of the steps of producing a frequency and/or signal intensity based data characterisation and inferring and outputting therefrom a result representative of the heart beat rate of the subject. In particular the data processing method involves deriving the result by identifying a peak in the frequency and/or by signal intensity based data characterisation for example by producing a power density spectrum, identifying the peak power frequency and, outputting the result as a result representative of the heart beat rate of the subject.

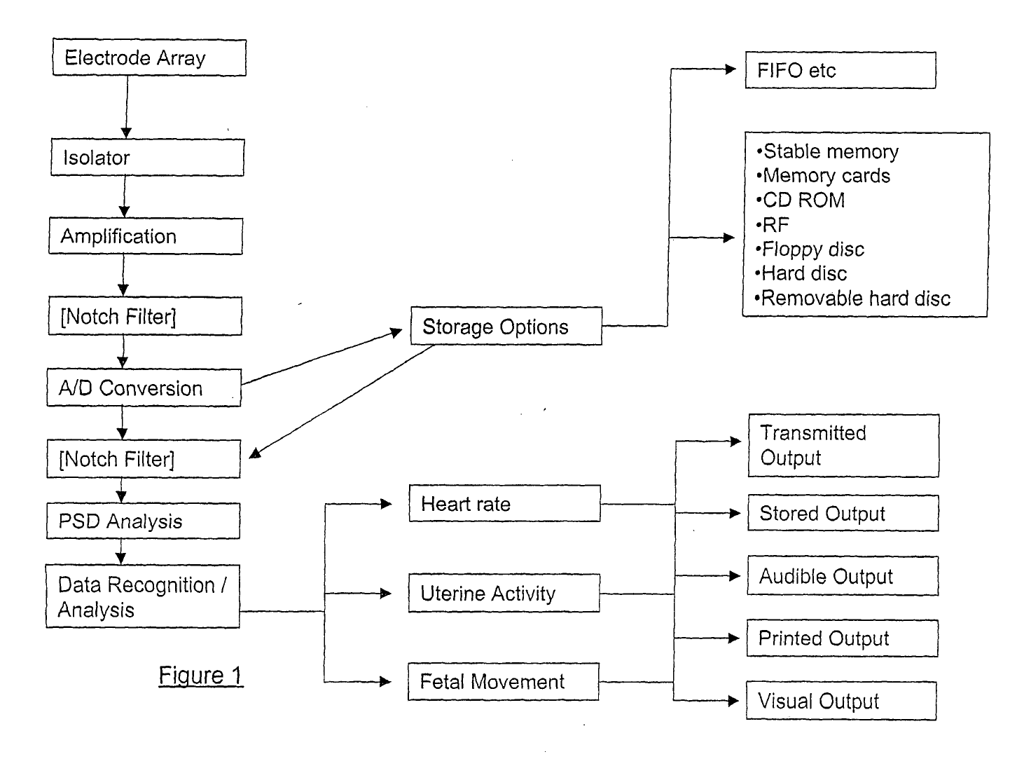

The invention relates to a heart monitoring device and method for monitoring the heart beat of a human or non human animal. The invention in particular relates to a non-invasive device and technique for the monitoring of heart rate. The invention in particular relates to the monitoring of the fetal heart of a human or non human animal by a non invasive technique.

The monitoring of the heart beat of a human or non human animal is of value in a wide range of circumstances for a wide range of medical or veterinary, and in particular diagnostic purposes.

In some circumstances it will be desirable to obtain a signal or measurement providing detailed information about the functional performance of the heart during one or more beat cycles, for example by use of electrocardiographic (ECG) techniques. Such techniques tend to require detailed skilled analysis of the results to produce an effective diagnosis, and tend to fall within the province of the skilled operative. In other circumstances, a relatively simple monitoring of heart rate or the like might be sufficient. This might be the case in particular in relation to regular monitoring of an ongoing condition, passive monitoring of a subject over a long test period etc. In these circumstances, a device and method which does not require a great deal of user expertise and which is relatively non-invasive and portable is likely to be preferred.

A particular example of the above arises in relation to routine fetal heart monitoring during the course of pregnancy. Other circumstances where a simple heart rate monitor might find particular applicability will also readily suggest themselves to the skilled person, for example in relation to devices to perform heart rate tests over a prolonged period on a subject, in relation to devices to monitor and provide warnings of abnormality in a subject with a chronic disorder etc.

Fetal heart monitoring is important to indicate the health of an unborn child, and takes increased significance during the labour process. In addition there can be significant social importance to the mother during pregnancy giving her confidence that nothing abnormal is occurring. Hence the implications and current techniques for fetal monitoring may be considered to fall into three categories: methods which fit in with the mother's lifestyle; methods which require some professional intervention (GP or midwife) but applicable to a domestic environment; and methods which require full clinical intervention, in particular in a hospital environment.

Socially, it is vitally important for the mother to build a relationship with her unborn child. Ways of doing this may be further split into mother- and GP-based measures.

The only mother-based measure is fetal movement. The mother starts to build a relationship with her baby from 16 to 20 weeks onward, and fetal movement is currently the only way of building this bond early.

The measure is highly subjective, variable during the day, and becomes different to the mother after 36 weeks. This can either cause the mother to worry unduly about a reduction in movement, or can lead to a mother carrying a dead fetus for some time before diagnosis. Both can obviously cause significant stress for the mother.

There are additional measures that may be used by skilled operatives (GP or midwife). The simplest technique is to listen to the fetal heart rate via a stethoscope. This needs to be skilfully positioned, and is unreliable in the accurate determination of fetal heart rate. Also the mother cannot hear the heartbeat and hence this does nothing to reassure and help build the relationship.

Portable Doppler devices are known which use ultrasonic waves to measure the heart rate. These can provide audible, visual and printable traces of heart rate. The audible output provides significant reassurance to the mother and helps to build a relationship with the developing fetus. However, the apparatus need to be positioned and operated carefully to get accurate results and therefore does not lend itself to unskilled operation.

In a case where there is a potential stillbirth, it may be difficult to distinguish the maternal and fetal heartbeats. Hence formal, hospital-based ultrasound scans are required for diagnosis in this case.

Additionally, in all stages of pregnancy fetal heart rate monitoring in a GP/Hospital environment may be used for diagnostic and intervention strategies. In this case the Doppler system is used extensively to deliver the following clinical evaluations:
1. The normal heart rate of a fetus ranges from 110 to 160 bpm during pregnancy. As fetal brain activity increases towards birth, the heart rate progressively drops. Generally an average fetal heart rate is 140 bpm, and if it drops below 100 bpm or above 160 bpm for more than 10 minutes then there is a potential for a significant problem with the fetus.
2. Before and during labour, the heart rate accelerates or decelerates and this information can be used to estimate the stress on the fetus. However deceleration measurement has a high false positive prediction of the rate of fetal distress.

The Doppler device has limitations and cannot be used if there is excessive movement of the baby. It may also be compromised if insonation of the fetal heart is hampered by adverse maternal body habitus such as obesity.

To reduce confusion, more direct techniques may be used during labour to determine fetal heart rate. For example the fetal ECG may be measured by a scalp electrode. This is attached to the baby in the labour process. It is obviously an invasive technique, rupturing the fetal membranes and requiring skill to insert a “cork-screw” type sensor into the vagina and attach to the scalp of the unborn baby. This provides a more accurate assessment of fetal heart rate as it measures R-R intervals and other changes in the PQRST complex (e.g. STAN analysis).

With either of the above it is common practice to measure uterine activity by palpation or via a force-sensing belt (tocodynamometer). This allows contractions to be measured and used along with heart rate monitoring to ascertain the condition of the baby before and during labour. With either method, there is considerable inter- and intra-observer variation in the assessment of uterine activity.

All the above techniques are now commonly and extensively used.

It may therefore be seen that:
1. Non invasive measurement of fetal heart rate, movement and contractions is a critical diagnostic tool that can determine fetal well being.
2. The process of developing the relationship between mother and baby can be greatly improved by “hearing” the heart rate of the baby as other self monitoring methods are unreliable.
3. All practical fetal heart rate monitoring systems need to be used by trained clinicians. In the Doppler device case, the system has limited reliability (unless it is a meterised device). Direct fetal ECG measurement is an invasive process and therefore confined to labour.

Several systems have been discussed that aim to give a degree of non invasive fetal heart rate monitoring. Previous investigators have described the indirect acquisition of the fetal ECG complex by means of skin electrodes on the mother's abdomen.

W00054650 describes a system of multiple electrodes placed externally on the mother to pick up both maternal and fetal heart rates. These are then subtracted and analysed via a complex method to determine fetal ECG. The device is not portable, and does not give audible output. It is designed to be used by a skilled operator.

U.S. Pat. No. 5,042,499 describes a system where two pairs of electrodes are placed externally on the mother. A skilled operator then manoeuvres one pair of electrodes around to adaptively cancel out the mothers' heart rate. The unit can then output instantaneous or beat to beat information but no audible output is provided.

W000126545 describes a system with 2 pairs of electrodes externally placed on the mother. It creates an ECG signal via signal processing but does not give instantaneous output. The device is designed to be portable, but analysed at a later date by a clinician.

Hence whilst systems are known that indirectly measure fetal heart rate, they rely on direct detection of the fetal PQRS complex and derivation of the fetal heart rate from R-R intervals. All are designed for skilled use and use complex algorithms to analyse the output signals. They do not generally produce instantaneous output and are not portable. Their accuracy is reliant on fetal position and their utility is therefore limited, given that this is constantly varying. They do not give audible output for mother use.

It is an object of the invention to provide a device for monitoring of a human or animal heart rate which mitigates some or all of the above disadvantages.

It is a particular object of the invention to provide a device and method which measures human or animal heart rate in simple and compact manner, using essentially non-invasive techniques, and in particular using techniques which require little or no skilled expert intervention for operation, and preferably also little or no expert intervention for diagnosis.

It is a particular object of the present invention to provide a device for monitoring the fetal heart rate, and in particular a non-invasive device which is compact and portable and which can be readily used by a non expert user.

Thus, according to the present invention in its first aspect there is provided a heart rate monitor apparatus including a monitoring part comprising a plurality of recording electrodes each incorporated into a contact surface for application to an external body surface of a human or animal subject and a means for acquisition of data from the electrodes, a data processing part comprising a means for conversion of acquired analogue to digitised data, a means for analysing the digitised data, optionally with reference to pre-recorded reference data and/or predetermined reference parameters, to produce an output, and optionally a display part to display a result corresponding to the output, wherein the means for analysing the digitised data includes means to process the data by way at least of the steps of producing a frequency and/or signal intensity based data characterisation and inferring and outputting therefrom a result representative of the heart beat rate of the subject. For example the result is derived by identifying a peak in the frequency and/or signal intensity based data characterisation. For example the means for analysing the digitised data includes means to produce a power density spectrum for the digitised data, identify the peak power frequency therefrom within a predetermined range corresponding to a range of possible heart beat rates, output the result as a result representative of the heart beat rate of the subject.

The analysing means especially includes means adapted to process the data by performing the steps of de-noising, signal isolation or conditioning the digitised data and/or performing a fast Fourier transform, wavelet transform or other mathematical transform on the digitised data to produce the said frequency and/or signal intensity based data characterisation (such as a power density spectrum) and derive the fetal heart rate.

The apparatus of the present invention thus incorporates body surface contactable electrodes and analyses a surface electrical signal to extract data concerning electrical cardiac activity. To that extent it bears similarities to conventional electrocardiographic techniques. However, the present invention is not designed to produce a full or partial ECG analysis/recognition. The present invention is adapted to infer the heart beat rate rather than produce a detailed electrical profile. The present invention relies on the surprising realisation that an effective indication of simple heart beat rate can be obtained without the need to resolve a full signal in the manner conventionally followed by ECG techniques.

In particular, in the prior art where conventional ECG techniques are used, it is necessary to resolve fully a QRS signal and to measure the beat rate by an analysis of the R waves therein. To get an effective measurement, a huge amount of extraneous data is processed, and the apparatus tends to be complex, large, and require operation by a skilled practitioner.

By contrast, in accordance with the invention, the electrical activity of the heart beat is not resolved and measured directly, but rather a representative “proxy” measure is obtained. The raw electrical activity data is acquired from the electrodes, and appropriate mathematical techniques are used to produce a frequency and/or signal intensity based data characterisation such as a power density spectrum. The heart rate is then inferred by interrogation of these data.

In contrast to the prior art, the present invention does not require a resolution of the detailed electrical cardiac activity to pinpoint R waves and to use these to measure heart rate. Instead, it relies on the surprising realisation that a perfectly effective rate measurement can be obtained merely by inference from a frequency and/or signal intensity based data characterisation, for example as a peak in the power density spectrum. The resulting apparatus can be greatly simplified, potentially made much more compact for home use, and potentially be available for non-expert application.

One or more reference electrodes may be provided in addition to the measuring electrode.

In a particularly preferred embodiment, four electrodes are provided in a bridge pair architecture.

The device thus includes a plurality of electrodes, each incorporated into a contact surface member for application to an external body surface of a human or animal subject. The contact surface may include a releasable adhesive strip. Each electrode may have its own contact surface. However, given the technique employed, precise positioning of electrodes is less critical that for a full ECG for example, and it then becomes practical for multiple electrodes to be provided with a common contact surface member.

The electrodes are thus preferably associated together in conveniently compact manner, for example being incorporated into a single common contact surface member, such as a single adhesive electrode strip for application to a body surface of the subject.

The apparatus provides a non intrusive apparatus for simple heart rate monitoring of a human or animal subject. Its simplicity of operation lends itself particularly to use by a non-expert user. In particular, the apparatus is suited to the measurement of fetal heart rate in non critical situations in non clinical environments, for example by the mother.

In those circumstances, the electrodes are adapted for application to the external abdominal surface of the mother in the uterine area. Data is acquired about electrical activity therein, which includes electrocardiac data from the fetus, and background noise caused for example by electrical activity in the uterine wall. The invention does not require characterisation of the ECG signal for the fetal heart rate. Rather, it is able to identify a result giving the fetal heart rate from frequency and/or signal intensity based data characterisation, for example as a peak in the power density spectrum, without the need for such detailed analysis. Additionally, it does not rely upon an accurate determination of fetal position. The invention is thus particularly suited to the measurement of fetal heart rate.

The monitoring part comprises a means to acquire a signal. The data processing part comprises a means to analyse the result. The parts may be fully or partly integrated and/or all or part of the data processing part might be separately provided and operably linked thereto. The optional display part may be similarly integral or separate. In non-integral embodiments components may be collocated or remote and may be wire or wirelessly linked for data communication. Components may comprise data storage means to store data for subsequent reading/analysis and/or transmission means to transmit the output to a remote site for immediate and/or subsequent reading/analysis of the results and/or data storage.

Preferably, the apparatus further comprises an optionally integral means to display the output result, in particular in a manner readily interpretable by a non-expert user. The display means may be adapted to provide any suitable display, including audible, printed, visual and combinations thereof. The display means may be adapted to give, for instance by means of an alphanumeric display, a direct reading of the measured heart rate.

Alternatively, for a number of applications it might be preferably that a direct reading is not given, but that the output result is compared with a set of predetermined reference parameters or predetermined values, and a display signal is generated indicating that the result falls in one of a number of discrete ranges. In such an embodiment the data analysis means includes a data storage comprising such predetermined reference parameters or predetermined values and a means to compare the output peak frequency therewith and generate a result based on that comparison.

For example, it might be desirable if the system is adapted to output results on a three state system comprising a heart rate in the normal range, a heart rate below the normal range, and a heart rate above the normal range. For fetal heart rate monitoring, the device is preprogrammed with a normal range which is preferably in the range 110 to 160 beats per minute.

In such a simple embodiment, the display means may comprise merely means to give a visual or audible warning if the heart rate falls outside the predetermined safe range. This simplifies still further the level of user skill required. Such a device may be adapted for use by a person with an established chronic tendency to experience accelerated heart rate. Suitably preprogrammed, the device might be worn permanently and allow an early warning to be given of accelerated heart beat, giving an indication to the subject that a period of rest would be in order. Similarly, a suitably preprogrammed device in accordance with the invention adapted to be worn by a pregnant mother could be set up to give a warning if the heart rate of the fetus fell above or below a predetermined safe range.

Comparison with pre-recorded data may be with data preprogrammed by default into the system, or subsequently and variably programmed by a medical supervisor, for example enabling detection of a particular condition. The data storage means thus comprises a suitable memory, programmable or permanent as the case may be.

The invention comprises a user worn device including at least the monitoring part of the invention (the electrodes and the means for data acquisition) compactly associated together to be easily worn by the subject, for example in the form of a single electrode patch. Since a detailed analysis of the electrical signals is not required in accordance with the invention the precise placing of different electrodes in not as critical as it would be for a conventional ECG device, and accordingly such a device, with simple instructions for a non-expert user, is adequate.

The device adapted to be so attached to and worn by the subject optionally further incorporates some or all of the signal processing means (being the means for converting analogue to digital data, the means for data analysis, and the means to output the results) and display if any. Any remote components may be directly wired to the subject contacting portion, or may be provided remotely in wireless communication therewith. In this latter case, the subject worn device is further provided with a transmitter adapted to operate on a suitable wireless protocol, and in particular a local short range RF protocol such as Bluetooth.

In such cases the invention further comprises the user worn part adapted for use with but provided separately from the remote part(s), and further comprises a kit of parts consisting of the user worn part and such remote part(s) as are necessary to effect functioning in accordance with the invention.

The device of the invention suitably comprises any means for operatively associating the assembly of components, at least forming the user worn part, such as a casing, mounting, cassette, card, frame or the like. A mounting may be rigid or flexible, for example a flexible mounting may comprise an article of clothing such as a belt, patch or the like which may be conveniently worn by the subject for prolonged periods without inconvenience.

The device may be manually operated or may comprise means for intermittent operation allowing periodic inspection on a regular basis.

Activation may be manual or automated for example as a periodic activation using a timer control means, and/or as a manual activation such as through a power switch. The device preferably incorporates a portable power supply such as a rechargeable or non-rechargeable battery. Additionally or alternatively means are provided to connect the device to a mains power supply.

Display means may be auditory, visual or both and conveniently indicates a result without the need for the subject to interpret levels or the frequencies of activity, i.e., in the form of a set of illuminating lights, tactile patterns such as vibration or discrete auditory signal or alarm or an alphanumeric display to display simple messages. In a multi-state embodiment such as the preferred three-state embodiment, multi-coloured lights may be used, each colour indicating a different condition (rate below a normal range, rate in the normal range, rate above a normal range).

Data acquisition and digitisation is suitably carried out by means of a microprocessor associated co-operatively with the electrodes using known techniques. Data analysis is by any suitable means and method associated with parameters related to change in electrical signals to produce a power density spectrum, and the analyser is adapted to perform such an analysis, for example by suitable programming.

The use may be human or animal use. In each case the device may be used for humans or animals in remote regions away from clinical supervision. Use of the device may be used within the home or in transit, during a subject's normal daily activities in public, or in the medical establishment. It is a particular advantage of the invention that the use is simple, convenient and the device may be operated by skilled or unskilled personnel, by the subject or by any other person.

In accordance with a further aspect of the invention there is provided the use of the foregoing device for diagnostic or monitoring purposes in relation to heart beat rate, especially for the monitoring of fetal heart rate, for the long term monitoring of a chronic condition, or for the passive heart rate monitoring over a prolonged test period.

In accordance with a further aspect of the invention there is provided a method for the measurement of heart rate, especially for the purposes of monitoring or diagnosis, comprising:

attaching a plurality of recording electrodes to an external body surface of a human or animal subject;

acquiring data therefrom corresponding to internal electrical activity measured at the surface;

immediately or subsequently processing the acquired data by:

digitising the data; analysing the data by producing a frequency and/or signal intensity based data characterisation; inferring and outputting therefrom a result representative of the heart beat rate of the subject.

For example the result may be inferred by identifying a peak in the frequency and/or signal intensity based data characterisation. More specifically for example the acquired data is analysed to produce a power density spectrum, identifying the peak power frequency is identified therefrom within a predetermined range corresponding to a range of possible heart beat rates; and the peak frequency result is output as a result representative of the heart beat rate.

Preferably, output is to or via suitable display means, in particular simultaneously.

In a preferred embodiment of the method, the method is a method for the measurement of fetal heart rate and comprises applying potential measuring electrodes to an abdominal surface of a mother in the uterine region, activating the device for a sufficient period to record electrical activity, acquiring the data and analysing in accordance with the foregoing to produce a result indicative of fetal heart rate.

The invention in accordance with further aspects comprises a computer program or a suitably programmed computer adapted to perform some or all of the above method steps, and in particular adapted to receive input data from a comprising digitised data corresponding to measurements of electrical activity on the body surface of a subject, process the data to produce a frequency and/or signal intensity based data characterisation, infer therefrom data representative of the heart beat rate of the subject, out put these data to an output register.

More specifically the program or computer is adapted to receive input data, analyse to produce a power density spectrum, identify the peak power frequency therefrom within a predetermined range corresponding to a range of possible heart beat rates, output the peak frequency result as a result representative of the heart beat rate to an output register.

The invention will now be described by way of example only with reference to the accompanying drawings which is a block diagram illustrating the mechanism of signal capture/analysis/display in an example according to the invention used to measure fetal heart rate.

The device uses for example two electrodes with or without a reference, or a four electrode array bridge (orthogonal electrode architecture) or up to six electrodes in an array. These are attached on the mother's abdomen. The siting of the electrodes is relatively unimportant and hence can be undertaken by a non-skilled person i.e. mother.

The output from these electrodes is fed via an isolation device to A/D converters where each signal is sampled within a range of 20 to 20,000 Hz concurrently to preserve phase information in the signal.

Acquired data may be temporarily stored within the device in the form of a dynamic constant motion non-stable memory. The storage device can be by RAM memory, dynamic buffer, FIFO, stable storage etc.

The signal is then de-noised by wavelet or any other appropriate algorithm and a suitable frequency or signal intensity based construct is formed for isolation of the fetal heart rate. In the example a Power Spectral Density (PSD) is constructed. The use of an adaptive notch filter dominant or digital filter, or a frequency based data filter prior to mathematical transform then allows the fetal heart rate peak to be identified. In a particularly preferred embodiment the method involves indirect identification of the fetal heart rate peak. The method is used to extract directly the much more dominant mother's heart rate peak. It has been found that the fetal heart rate peak, although much less intense, is closely adjacent thereto. Thus, it is easily located once the larger mother's heart rate peak is characterised. In accordance with the method the fetal heart rate peak is then easily extracted. Peak characteristics such as band width, skew of bandwidth, shape and form both mathematically and user defined, can be used for the purpose of fetal behaviour monitoring and characterisation.

The fetal heart rate is sharply discriminated in the example case because of the closeness of the sensing to the fetus, and the use of a bridge or reference electrode to remove other mother driven electrical signals. The use of four electrodes in a bridge also allows high gains to be generated increasing signal quality.

The resulting real time signal can then be used to:
1. Trigger an audio output synthesised from fetal heart recordings as an indicator
2. Be logged over time in onboard memory to be later analysed for trends or events
3. Be output visually by means of a flashing indicator, wave form, or signal alarm
4. Be output as traces
5. Be further analysed to give acceleration/deceleration data, RR, RR power spectral densities, and any other analysis require for optimal diagnosis.
6. This device can be used from second trimester to delivery.

Additionally, the analysis method may not be restricted to just fetal heart rate. Other peaks in the PSD may be analysed to give information on uterine activity and baby movement. Peak characteristics such as band width, skew of bandwidth, shape and form both mathematically and user defined, can be used for the purpose of fetal behaviour monitoring and characterisation.

1. A heart rate monitor apparatus including a monitoring part comprising a plurality of recording electrodes each incorporated into a contact surface for application to an external body surface of a human or animal subject and a means for acquisition of data from the electrodes, a data processing part comprising a means for conversion of acquired analogue to digitised data, a means for analysing the digitised data to produce an output, and optionally a display part to display a result corresponding to the output, wherein the means for analysing the digitised data includes means to process the data by way at least of the steps of producing a frequency and/or signal intensity based data characterisation and inferring and outputting therefrom a result representative of the heart beat rate of the subject.

2. Apparatus in accordance with claim 1 wherein the means for analysing the digitised data includes means to derive the result by identifying a peak in the frequency and/or by signal intensity based data characterisation.

3. Apparatus in accordance with claim 2 wherein the means for analysing the digitised data includes means to produce a power density spectrum for the digitised data, identify the peak power frequency therefrom within a predetermined range corresponding to a range of possible heart beat rates, output the result as a result representative of the heart beat rate of the subject.

4. Apparatus in accordance with claim 2 wherein the analysing means includes means adapted to process the data by performing the steps of de-noising, signal isolation or conditioning the digitised data and/or performing a fast Fourier transform, wavelet transform or other mathematical transform on the digitised data to produce the said frequency and/or signal intensity based data characterisation and derive the fetal heart rate.

5. Apparatus in accordance with claim 1 wherein the analysing means includes means adapted to process the data with reference to pre-recorded reference data and/or predetermined reference parameters.

6. Apparatus in accordance with claim 1 wherein four recording electrodes are provided in a bridge pair architecture.

7. Apparatus in accordance with claim 1 wherein one or more reference electrodes are provided in addition to the recording electrodes.

8. Apparatus in accordance with claim 1 wherein the contact surface of each electrode includes a releasable adhesive strip for application to a body surface of the subject.

9. Apparatus in accordance with claim 1 wherein multiple electrodes are provided with a common contact surface member.

10. Apparatus in accordance with claim 9 wherein the electrodes are associated together in conveniently compact manner being incorporated into a single common contact surface member for application to a body surface of the subject.

11. Apparatus in accordance with claim 1 further comprising a display to display the output result, in particular in a manner readily interpretable by a non-expert user.

12. Apparatus in accordance with claim 11 wherein the output result is compared with a set of predetermined reference parameters or predetermined values, and a display signal is generated indicating that the result falls in one of a number of discrete ranges.

13. Apparatus in accordance with claim 12 wherein the display comprises merely means to give a visual or audible warning if the heart rate falls outside a predetermined safe range.

14. The use of a device in accordance with claim 1 for diagnostic or monitoring purposes in relation to heart beat rate, especially for the monitoring of fetal heart rate, for the long term monitoring of a chronic condition, or for the passive heart rate monitoring over a prolonged test period.

15. A method for the measurement of heart rate, especially for the purposes of monitoring or diagnosis, comprising:
attaching a plurality of recording electrodes to an external body surface of a human or animal subject;
acquiring data therefrom corresponding to internal electrical activity measured at the surface;
immediately or subsequently processing the acquired data by:
digitising the data; analysing the data by producing a frequency and/or signal intensity based data characterisation; inferring and outputting therefrom a result representative of the heart beat rate of the subject.

16. The method of claim 15 wherein the result is inferred by identifying a peak in the frequency and/or signal intensity based data characterisation.

17. The method of claim 16 wherein the acquired data is analysed to produce a power density spectrum, identifying the peak power frequency is identified therefrom within a predetermined range corresponding to a range of possible heart beat rates; and the peak frequency result is output as a result representative of the heart beat rate.

18. The method of claim 15 adapted for the measurement of fetal heart rate and comprising applying potential measuring electrodes to an abdominal surface of a mother in the uterine region, activating the device for a sufficient period to record electrical activity, acquiring the data and analysing to produce a result indicative of fetal heart rate.

19. The method of claim 18, wherein the step of analysing the data to produce a result indicative of fetal heart rate comprises the steps of producing a power density spectrum in which heart rate peaks can be identified, identifying from the power density spectrum the mother's heart rate peak, locating therefrom the smaller fetal heart rate peak, is closely adjacent thereto, outputting the located peak frequency as a result indicative of fetal heart rate.

20. A computer program or a suitably programmed computer adapted to perform some or all of the method steps in accordance with claim 15.

21. A computer program or a suitably programmed computer in accordance with claim 20 adapted to receive input data from a comprising digitised data corresponding to measurements of electrical activity on the body surface of a subject, process the data to produce a frequency and/or signal intensity based data characterisation, infer therefrom data representative of the heart beat rate of the subject, out put these data to an output register.

22. A computer program or a suitably programmed computer in accordance with claim 21 adapted to receive input data, analyse to produce a power density spectrum, identify the peak power frequency therefrom within a predetermined range corresponding to a range of possible heart beat rates, output the peak frequency result as a result representative of the heart beat rate to an output register.
